(19) United States
(12) Patent Application Publication
(10) Pub. No.: US 2010/0087713 A1
(43) Pub. Date: Apr. 8, 2010

(54) Shapeable pad

(76) Inventor: Chaim Eliash, Ya'akov (IL)

Correspondence Address: NIXON & VANDERHYE, PC, 901 NORTH GLEBE ROAD, 11TH FLOOR, ARLINGTON, VA, 22203, US

(21) Appl. No.: 12/310,813

(22) PCT Filed: Sep. 5, 2007
(86) PCT No.: PCT/IB2007/053586

§ 371 Date: Dec. 8, 2009

Related U.S. Application Data
(60) Provisional application No. 60/842,952, filed on Sep. 8, 2006.
(60) Provisional application No. 60/935,564, filed on Aug. 20, 2007.

(51) Int. Cl. A61B 1/32

(52) U.S. Cl. 600/206

(57) ABSTRACT

The present invention provides a device comprising a plastically deformable or elastically deformable core component embedded within a pad, sheet or drape. In addition, the present invention further provides methods for the hands-free retraction of organs and tissues during surgical and dental procedures and for the protection of organs and tissues during such procedures.

### FIELD OF THE INVENTION

The present invention relates to deformable pads and similar devices that may be used primarily as surgical retractors. The pad device of the present invention may also be used in other, non-surgical applications.

### BACKGROUND OF THE INVENTION

Surgical procedures require retraction and separation of organs and tissues. Such retraction is fundamental to the surgeon's ability to perform the procedure. Poor retraction and inadequate separation of tissues result in poor visualization, thereby providing a sub-optimal environment for the surgical team to perform its work.

The surgeon is often required to expose and handle delicate tissues deep within the body cavities. This requires opening a surgical window, or workspace, through which the procedure can be performed. The surgical window should be optimized such that it is wide enough to view and work within the treatment area, while taking care not to damage the surrounding tissue.

Retractors consisting of a blade and a handle held by a nurse or an assistant surgeon have been used in surgery for centuries. More recent retractors may be attached to the operating table or to some other external object using various methods, and maintain the desired tissues and/or organs in a position dictated by the surgeon.

Retraction is widely needed in every kind of surgery. While there are retractor devices on the market with the ability to retract tissues and create the surgical space in various ways, most retractors fail to retract soft tissues, especially when the designated area of retraction is deep in the body cavity. For example, abdominal and pelvic surgery requires wide retraction of the abdominal wall.

However, whereas most currently-used retractors are able to retract the abdominal wall and the upper part of the intestine, they fail to retract the lower part of the intestine. As such, one surgeon's hand would be fully devoted to holding part of the intestine and preventing it from falling into the surgical space. Since the intestines are too sensitive for rigid and traumatic mechanical attachment, and might be damaged by normal retractors, such retractors are not suitable for use with this organ.

The same applies to other areas of the body where the procedure in question is being performed in a deep, narrow cavity, making it difficult for normal retractors to open the area, and produce a separation of tissues. A good example of this is the lymphatic lateral axillary dissection procedure. The use of standard prior art retractors in this type of surgery to retract tissues and isolate a surgical space is problematic because of a deep and narrow surgical operation field.

Additionally, throughout the human body various important vessels enclose pressurized streams of biological fluid, for example blood (e.g. in the aorta and other blood vessels) or urine (in the ureter.) It is not uncommon for a surgeon to accidentally injure a vessel by a miscalculated move of a surgical tool. Such injuries can be fatal, since often the injury is small and unnoticeable, and the patient's condition deteriorates only post-operatively. To avoid such injuries, the surgeon must locate the vessels at the beginning of surgery, prior to dissecting. Once the vessel is located, it is marked clearly with suture threads. Although marked, in some instances, in the course of the focus on the surgery site, the surgeon may accidentally injure the vessel. Shielding these vessels from injury is therefore of primary importance.

There therefore exists a well-recognized need for a simple sterilizable instrument or device that has the ability to retract tissues, absorb fluids, and shield organs, vessels, and soft tissues from injury during surgery.

### SUMMARY OF THE INVENTION

It has now been unexpectedly found that it is possible to construct a biocompatible, sterilizable pad device having the ability to be either plastically or elastically deformed in three dimensions, in any direction and along any axis, such that said pad device is suitable for use as a customizable surgical retractor. Moreover, it has been found that following deformation (e.g. bending or folding) of said pad device, it may be returned to a conformation identical or very similar to its original conformation and subsequently bent or folded into a new conformation. As a result of this property, the device may be moved and re-folded several times during the course of a surgical procedure, in order to adjust the retraction provided thereby or in order to retract tissues or organs in another part of the surgical field.

For the purposes of the present disclosure the term “plastically deformable” (and the like) is to be understood as referring to the ability of a pad device of the present invention to remain in the three-dimensional conformation into which it is brought by means of manual deformation. Conversely, the term “elastically deformable” (and other, similar terms) refers to the ability of the pad to resume or to attempt to resume its initial (resting) state after the manual force which has caused its deformation has been removed.

Thus, in its most general form, the present invention is primarily directed to a pad, sheet or drape having the ability to act as a hands-free surgical retractor (i.e. to move surgical tissues and organs to a new position and or hold said tissues and/or organs in that new position), and to shield organs, vessels, and soft tissues from injury.

In a particularly preferred embodiment of the invention, the pad is constructed of a material such that it is able to absorb fluids (such as blood) present at the surgical site.

In its most general form, the device of the invention is to be considered as comprising two components:
a) a pad, sheet or drape of woven or non woven material, embedded in which is:
b) a deformable core component comprising at least one element which is either plastically deformable or elastically deformable.

For the purpose of the description and claims that follow the term ‘pad’ will be used as a generic term to include other soft woven and non-woven elements such as sheets and drapes.

In one particularly preferred embodiment, the device of the invention is constructed such that the deformable core component is present across the entire width and length of the pad element. In other preferred embodiments, the core component occupies less than 100% of the area of the pad element. In one preferred embodiment the core component occupies more than 30% of the surface area of the pad. In another preferred embodiment, the core component occupies more than 50% of the surface area of the pad. In yet another preferred embodiment, the core component occupies more than 70% of the surface area of the pad.

In one preferred embodiment, the device of the invention consists of a gauze pad or other absorbent element and at least one plastically deformable element capable of being shaped or formed into a stable conformation. The device is capable of repeatedly undergoing three dimensional, multi-directional deformations without rupture or relaxation.

In another embodiment, the device of the invention consists of at least one absorbent element (e.g. a gauze pad) and at least one elastically deformable element. The elastically deformable element is capable of returning to an initial form or state after deformation, and is capable of being rotated along three-dimensional and multi-directional axes.

In other preferred embodiments of the device the pad component is constructed of non-absorbent materials such as polystyrene, or rubber, or of an absorbent material coated with a non-absorbent coating such as a polyamide, silicon or other suitable polymer coating.

The deformable core component of the device of the present invention may be constructed in several different ways. Thus the present invention includes within its scope core components constructed as matrices formed from series of parallel strips or rods as well as solid plates which may optionally be perforated by a series of holes or slots. Details of exemplary core components of the present invention are described in more detail hereinbelow and illustrated in the accompanying drawings.

During surgical operations the pad device may be inserted into a surgical field in order to retract or hold apart tissues or organs which may otherwise obstruct the surgeon's view, thereby allowing the production of a better working space for the surgeon. The surgeon can then place and sculpt the pad, forming it into a structure which will hold the organs and tissues ad hoc. In the plastically deformable embodiment, the present invention has a self-form memory, which retains the shape once it is set, and does not require external force (such as clips or other forms of attachable elements) to maintain its position.

Consequently, in one aspect, the present invention is directed to a method for hands-free retraction of organs and tissues during surgery comprising the steps of:
a) providing a pad device as disclosed hereinabove and described in more detail hereinbelow;
b) bending the pad device to the desired shape; and
c) inserting the pad device into the desired location within the surgical field, thereby causing the retraction of the tissues and organs present at said location;
   wherein steps (b) and (c) may be performed in any order.

In addition to its use as a retractor in surgical procedures that are carried out in the operating room, the present device may also be similarly used to retract soft tissues during dental procedures. Typical examples of such an application would include the outward retraction of the labial mucosa and the lateral retraction of the buccal mucosa. Clearly, the present invention, with its ability to be precisely molded into any desired shape and to retain that shape in a hands-free manner, offers several advantages over the cotton wool rolls and dental mirrors which are often currently used for retracting these tissues.

Another use of the invention is as a soft, protective shield, having a hard (for example, metallic or plastic) internal core component, with the ability to protect the tissues, blood vessels and other structures from accidental injury by surgical working tools. Consequently, the present invention further provides a method for protecting organs and tissues during surgery comprising the steps of:
a) providing a pad device according to claim 22;
b) bending the pad device to the desired shape; and
c) positioning the pad device around the organ or tissue to be protected;
   wherein steps (b) and (c) may be performed in any order.

The abovementioned method may be used to protect any desired tissues, organs or structures during surgery, including (but not limited to) tubular structures such as blood vessels, various other body passages such as the ureter and trachea, as well as solid organs exposed during surgery within the abdominal cavity and elsewhere.

Typically, the device may be constructed out of a plastic or metal skeleton, which is covered on both sides by a pad, sheet or drape material which may be either absorbent or non-absorbent, and in either case may be woven or non woven.

The main advantage of the device of the present innovation, as compared to prior art devices, is its multifunctional nature: thus, the present device is soft and cushioning, as well as absorbent, and at the same time adjustable to any shape, with “shape memory” along all parts of the pad. Hence, the device can serve to retract or hold soft tissues and\or serve the other functions of a surgical pad, such as absorbing liquids or protecting tissue. This unique combination of qualities imparts some further exceptional advantages to the present invention in comparison with prior art retractors or pads.

These advantages include:
Ability to absorb fluids: In many instances, the surgical field does not allow for the insertion of a large number of surgical tools. A single device—i.e. a dual-function pad—provides a solution to this problem. While retracting, the pad of the present invention, also “serves” as a regular surgical pad. Prior art retractors do not have this large surface area absorptive property.
Cushioning and shielding: The “pad-sponge” configuration of the invention allows the device to retract soft tissues in a trauma-free manner, while the rigid inner portion allows the device—in addition to its function as a conventional absorbent pad—to protect organs, vessels, and/or tissues from accidental injury. The trauma-free nature of the presently-disclosed device is related to at least two of its structural features: the use of a soft material to cover or coat the otherwise sharp edges of the deformable core component, and the high degree of softness of the pad. These two features will be discussed in more detail hereinbelow.
Shapeability: While other pads cannot be positioned and retained in a designated area without further anchoring, the present invention can secure itself in the designated area in a pre-set position and shape, as desired by the surgeon, thus allowing the presently disclosed pad to act as a “hands-free” device. The fact that the device of the present invention is provided in the form of a pad or sheet (i.e. having two opposing faces of identical size and shape, wherein the ratio of the surface area of each face to the thickness of the device is very large) permits the device to be manipulated into a significantly greater number of shapes, sizes and forms than prior art devices which are provided in the form of elongate rods or assemblies of a small number of such rods. For example, a single device of the present invention may be used such that effectively the entire surface area of one face of the pad is in contact with the retracted organs. Alternatively, it may be folded several times until it adopts a different topology e.g. spheroid, rod-like, irregular three-dimensional shape etc. In other words, the fact that the device is provided in the form of a pad or sheet enables a single device to be brought into an essentially unlimited number of different conformations, in accordance with the surgical requirements. This difference between the presently-disclosed pad device and the elongate devices of the prior art is related, at least in part, to the fact that while the former is essentially non-directional in that it may be bent and folded along any axis, the elongate devices may be manipulated in a restricted number of axes only (e.g. it is not possible to fold a rod-shaped device about its longitudinal axis).

In summary, the device of the invention has the advantage of simplifying surgical procedures, saving the surgeon's time, freeing the surgeon's hand, and improving patient safety.

All the above and other characteristics and advantages of the present invention will be further understood from the following illustrative and non-limitative examples of preferred embodiments thereof.

### BRIEF DESCRIPTION OF THE DRAWINGS

The present invention is illustrated by way of example in the accompanying drawings, in which like references indicate similar elements and in which:

FIG. 1 schematically illustrates an exemplary embodiment of the deformable core component of the invention;

FIG. 2 schematically illustrates another exemplary embodiment of the deformable core component of the invention;

FIG. 3 schematically illustrates another exemplary embodiment of the deformable core component of the invention;

FIG. 4 schematically illustrates another exemplary embodiment of the deformable core component of the invention;

FIG. 5A schematically illustrates a side view of one exemplary embodiment of a deformable core component for use in the construction of the present invention;

FIG. 5B schematically illustrates a side view of another exemplary embodiment of a deformable core component;

FIG. 5C schematically illustrates a side view of yet another exemplary embodiment of a deformable core component;

FIG. 5D schematically illustrates a side view of a further exemplary embodiment of a deformable core component;

FIG. 5E schematically illustrates a side view of a still further exemplary embodiment of a deformable-adjustable core component;

FIG. 6 schematically illustrates a perspective view of some exemplary embodiments of various deformable core components of the present invention having different cross-sectional shapes;

FIG. 7 A-C schematically illustrates exemplary embodiments of various possible structures of the deformable core component;

FIG. 8 A-B schematically illustrates further exemplary embodiments of various structures of the deformable core component;

FIG. 9 schematically illustrates the multi-directional deformation of the present invention;

FIG. 10 schematically illustrates an exemplary use of one of the embodiments of the invention during abdominal surgery;

FIG. 11 schematically illustrates another exemplary use of one of the embodiments of the invention to protect major blood vessels and create a clear surgical field during surgery;

FIG. 12 schematically illustrates another exemplary use of one of the embodiments of the invention 1 as a means of protecting the ureter 7 during surgery;

FIG. 13 is a photograph of the invention retracting a pig intestine while the invention is positioned in a ‘Z’-shaped formation;

FIG. 14A is a photograph of the invention retracting a pig intestine;

FIG. 14B is a photograph of the same as in FIG. 14A after the device of the invention has been removed;

FIG. 15A schematically illustrates the elastic embodiment in its closed position (Cesarean embodiment);

FIG. 15B schematically illustrates the elastic embodiment in its open position (Cesarean embodiment);

FIG. 16 schematically illustrates the use of the plastic embodiment in a biliary procedure;

FIG. 17 schematically illustrates the use of the plastic embodiment in a sigmoidectomy procedure;

FIG. 18 schematically illustrates the use of three separate devices in a urological surgical procedure;

FIG. 19 schematically illustrates the use of a plastic embodiment in a laparotomy procedure;

FIG. 20 depicts a stainless steel matrix suitable for use in the manufacture of the device of the present invention;

FIG. 21 shows the manufacturing stage during which silicon tubing is adhered to the outer border of the matrix;

FIG. 22 shows a matrix fitted with silicon tubing around its outer border;

FIG. 23 depicts pouches formed from a gauze pad;

FIG. 24 depicts an internal pouch formed from six-ply gauze;

FIG. 25 shows a six-ply gauze external pouch being fitted around the internal pouch;

FIG. 26 shows the device of the invention following additional sewing along its entire border;

FIG. 27 illustrates the combined use of two separate elastic versions of the present invention, in their open position during a Cesarean section procedure.

### DETAILED DESCRIPTION OF PREFERRED EMBODIMENTS

The present invention, as disclosed hereinabove and claimed hereinbelow, provides an improvement upon current surgical pads in both structure and method, and further combines the functionality of such pads with the functionality of surgical retractors. The surgical window produced during surgical procedures can be optimized (from the point of view of clarity of vision) by the use of a tissue retraction pad of the invention that may be inserted and placed into the surgical treatment area. The pad's malleable characteristics allow the surgeon to shape it as desired at the surgical site. As mentioned hereinabove, a key advantage of the pad device present invention is the fact that it may be shaped and bent in any direction, along any axis, and indeed along multiple axes at different points along the surface of the device. This multi-directional feature is schematically illustrated in FIG. 9 which depicts the manner in which a pad device of the present invention may be folded in several different directions, in an irregular manner, in order to suit the particular conditions prevailing at the operating site. The pad device will then remain stationary, retaining its shape and retraction capability. The characteristics of the device of the invention result from a combination of materials, structure, and shape.

As explained hereinabove, the pad retractor of the present invention is capable (in some embodiments) of functioning as a liquid-absorbing pad (in addition to its retractor function). Since the absorptive capacity of the absorbent embodiments of the pad retractors is a property of the pad (rather than the core component), this ability will be in part determined by the amount of absorbent gauze present in the device. In one preferred embodiment, the weight of the padding and absorbing material in the the absorbent version of the device of the invention is at least 35% of the total weight of the device. In another preferred embodiment of the invention the weight of the pad is at least 70% of the total weight of the device.

Further details concerning the absorptive capability of the pad retractor of the present invention are provided hereinbelow.

The pad device may be manufactured in two main formats. The first exemplary format will be referred to hereinafter as the ‘PLASTIC EMBODIMENT’, and the second exemplary format of the device will be referred to hereinafter as the ‘ELASTIC EMBODIMENT’.

In order to best explain the various embodiments of the invention, a few illustrative examples will now be brought. The purpose of these examples is only to exemplify the various embodiments; they are not intended, however, to limit the scope of the present invention to these embodiments alone.

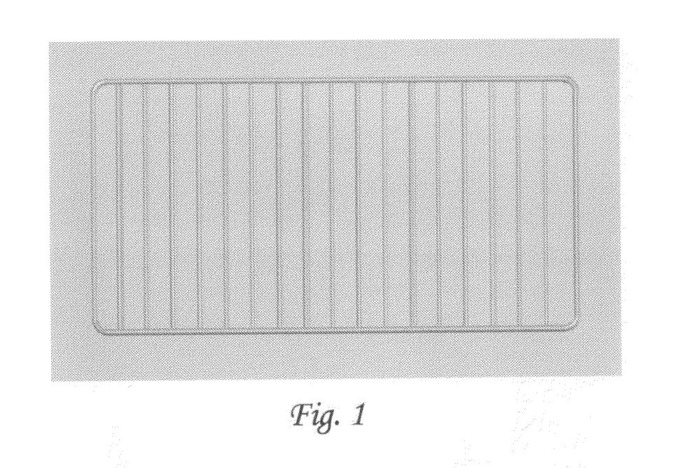

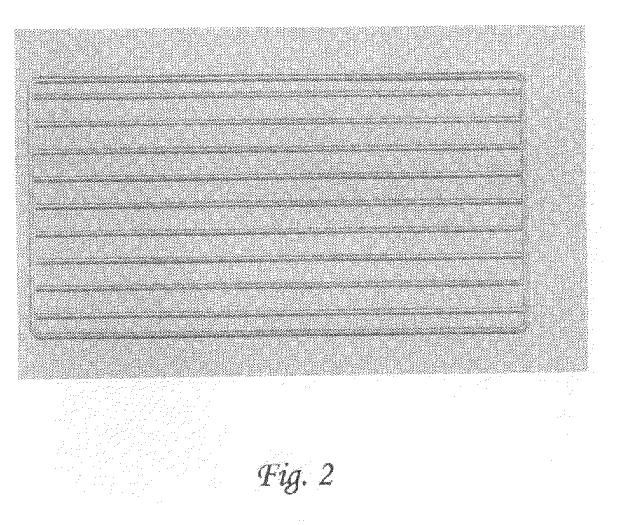

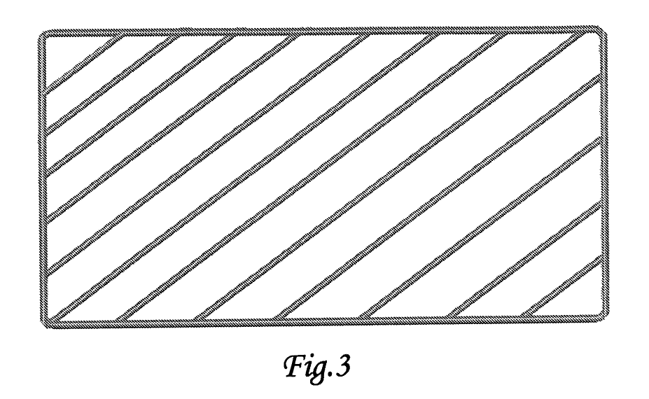

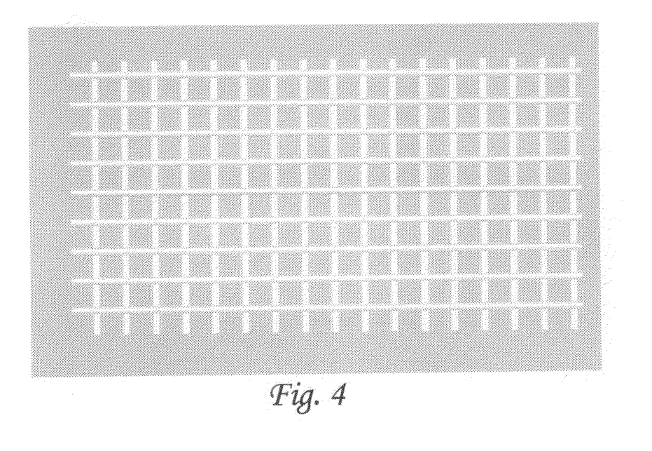

In one preferred embodiment of the PLASTIC EMBODIMENT of the invention, the device is comprised of a plastically deformable element constructed as a strip or series of strips. The strips are spaced apart from each other, and may be placed lengthwise and crosswise (as described in FIGS. 1 through 4), or may be set only lengthwise, or may be set only crosswise. In one preferred embodiment, the width of the strips is in the range of 3 to 10 mm. Preferably, the thickness of the strips is in the range of 0.5 to 3 mm.

The longitudinal and horizontal strips may or may not be attached to one another to form a two-dimensional matrix (e.g. a net). The strips are then covered from all sides with an absorptive material such as a sponge or gauze pad or a non woven pad. The pad gives the device the ability to absorb fluids from the surrounding operating area as well to serve as a cushion for the incision and surrounding surgical area, protecting it against injury from the various surgical instruments used to open the abdominal wall and perform the desired surgical operation.

In certain other embodiments, the deformable strips are covered by a non-absorbent material.

The inner plastically deformable element gives the device its ability to be shaped and reshaped in multiple directions and to remain in a steady position, as well as allowing the pad to be sculpted in the desired shape to allow retraction and\or tissue protection in accordance with the specific variable anatomical configuration and the surgeon's operative needs. These characteristics give the device its ability to act as a “stand alone” (or “hands-free”) self-retaining pad retractor with no need for securing elements attached to an external frame. This ability of the pad retractor of the present invention to be self-retaining is a function of two physical characteristics of the device: high flexibility of the device, on the one hand, and a strong retraction capability on the other hand.

The same embodiment may also be produced using one or more variations of the inner plastically deformable element described hereinabove, as exemplified below.

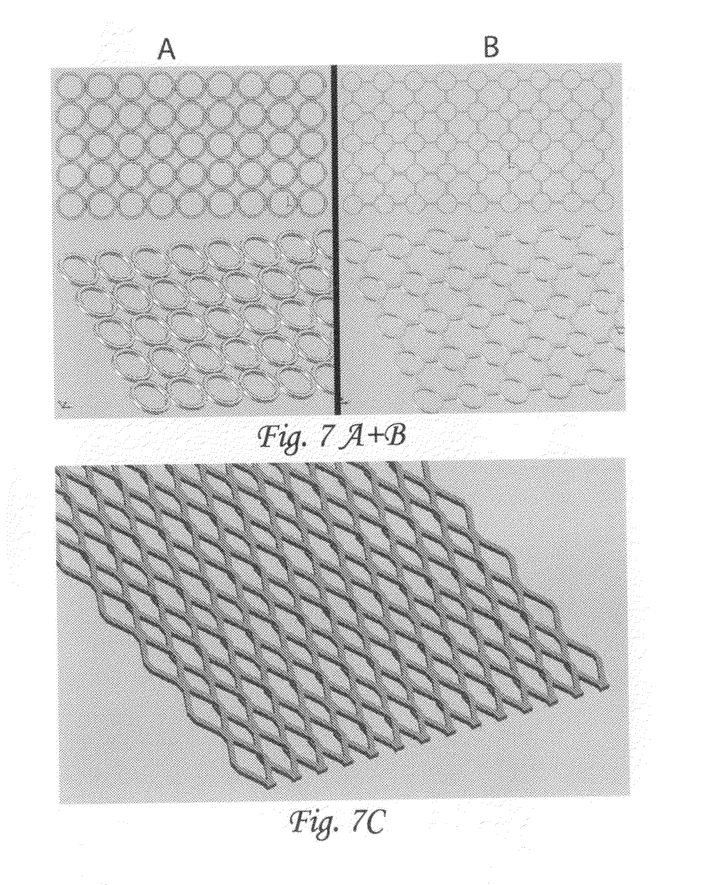

Thus, the basic element of the inner plastically deformable element may be constructed as a small round ring (2-7 mm) and connected to other such rings by strings or cords, or by any other method, thereby forming both a longitudinal and a horizontal net, as depicted in FIG. 7B

Alternatively, the inner plastically deformable element may be provided as a plurality of small three-dimensional rings (balls or spheres), all connected to one another, thereby forming a three-dimensional line, and both a longitudinal and a horizontal net, as illustrated in FIG. 7A. A further example of a net-like matrix (containing diamond-shaped apertures) is illustrated in FIG. 7C.

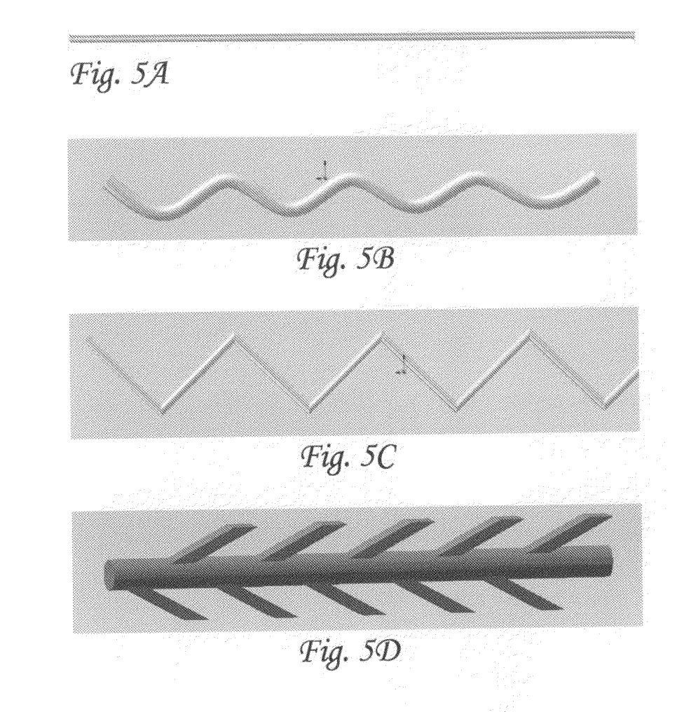

The plastically deformable strips may be formed in all shapes and dimensions, the lines may be straight (FIG. 5A), curved, wavelike (FIG. 5C and FIG. 5B), curled (FIG. 5E), or any combination thereof (e.g. as depicted in FIG. 5D), or in any other desired geometric shape.

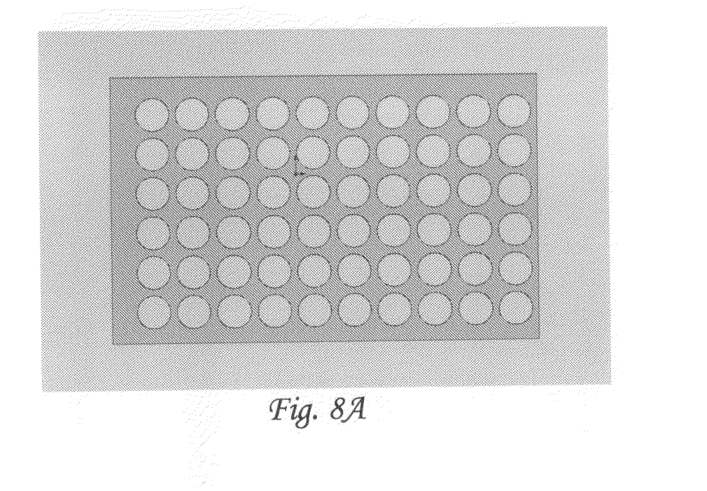

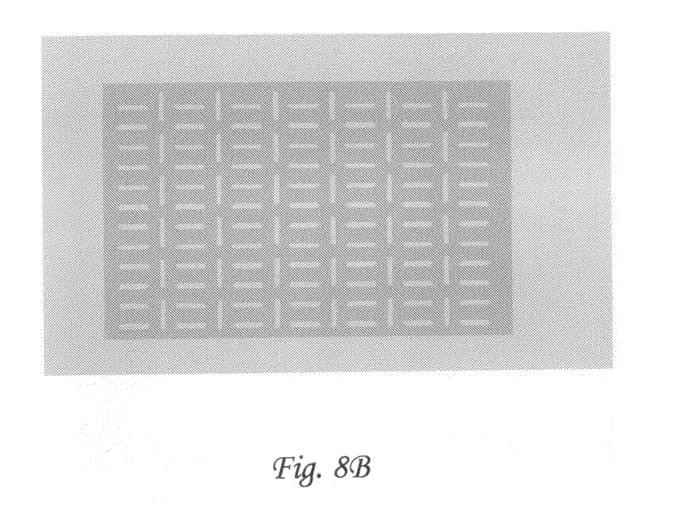

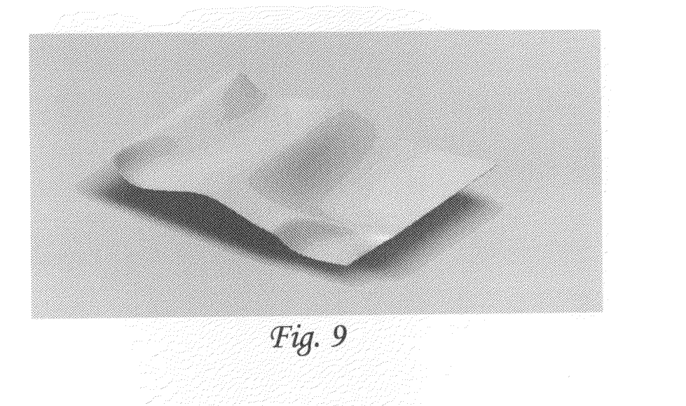

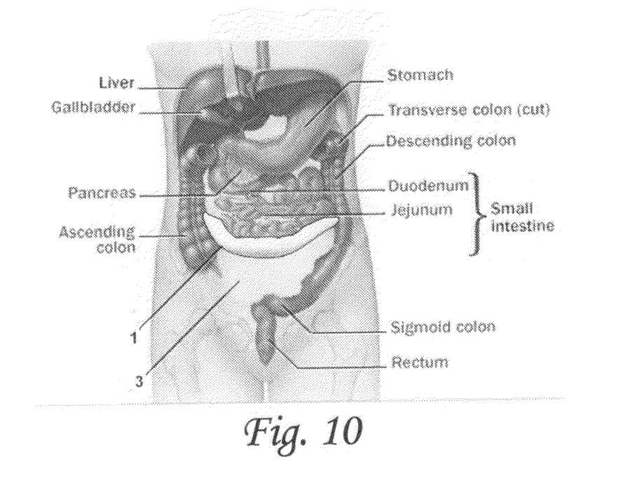

In an alternative embodiment, the plastically deformable element may be designed as a plate or as a plate with lines of holes or apertures, both longitudinal and horizontal. The holes may be cut in various sizes, or may be all of one size (FIG. 8A). Said holes can be made, for example, using laser cutting technology. The apertures need not be restricted to round holes, but rather may be formed in any convenient shape, for example as a series of slots as depicted in FIG. 8B.

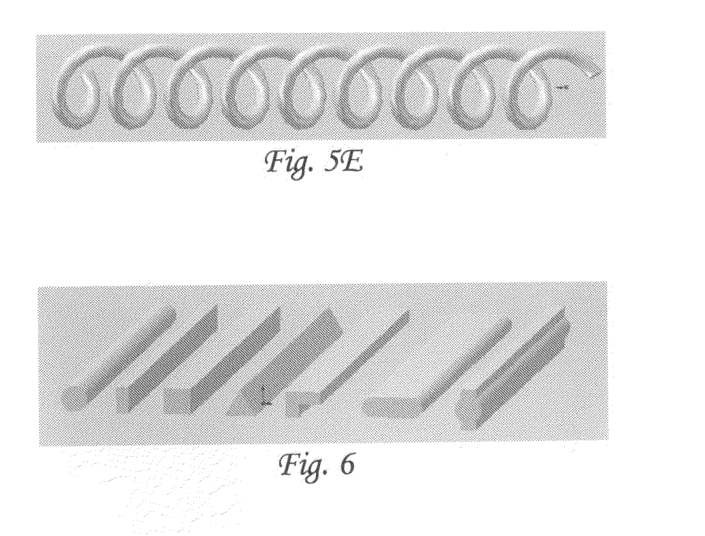

FIG. 6 illustrates a cross-section view of some exemplary geometric shapes of the plastically deformable strips.

### EXAMPLES OF USE OF THE PLASTIC EMBODIMENT

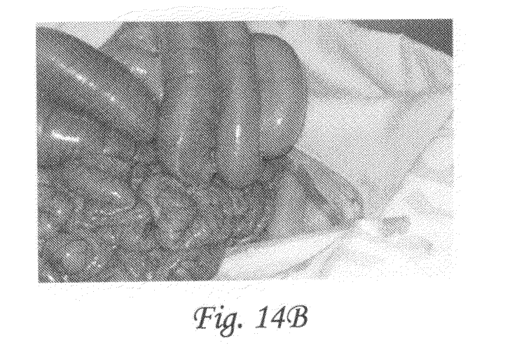

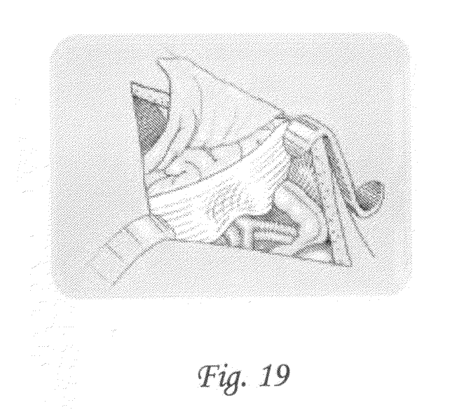

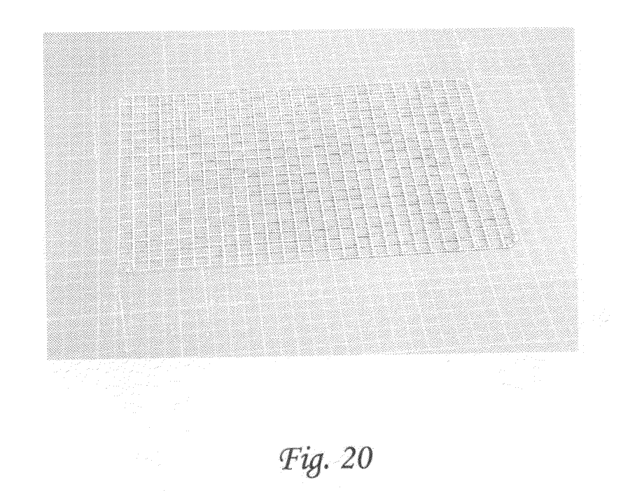

In the plastic embodiment, the invention may be inserted between the two vertical abdominal walls, creating another “wall” perpendicular to the abdominal wall, parallel to the intestine. The device then supports itself in its position against the abdominal walls, given the nature of the inner plastically deformable element, which maintains its form after it is shaped. The device serves as a barrier to prevent the intestine from collapsing into the surgical field, as shown in FIG. 14A (a photograph depicting the use of the device to retract the intestines of a pig while self-securing its positioning, with no need for any external support means). This application of the device of the present invention is also illustrated in FIG. 19. FIG. 14B demonstrates the collapse of the intestine into the surgical field when the device is removed).

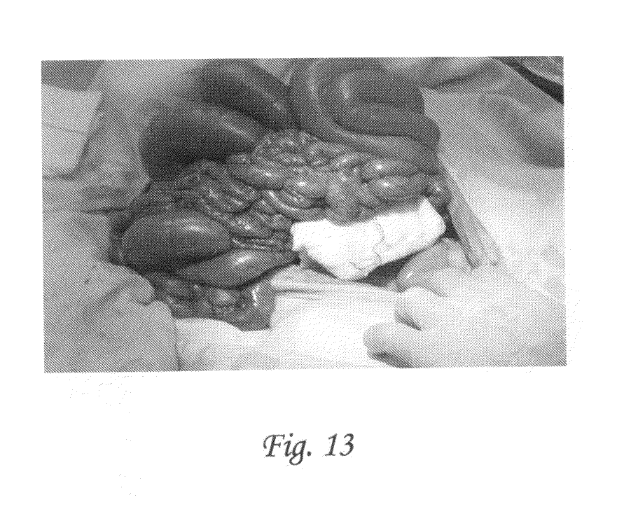

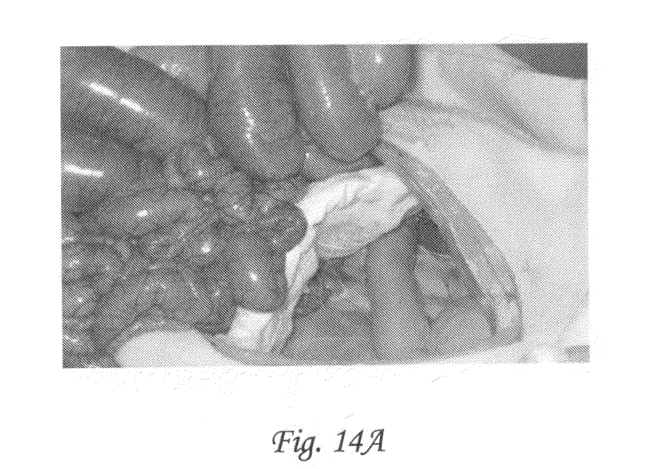

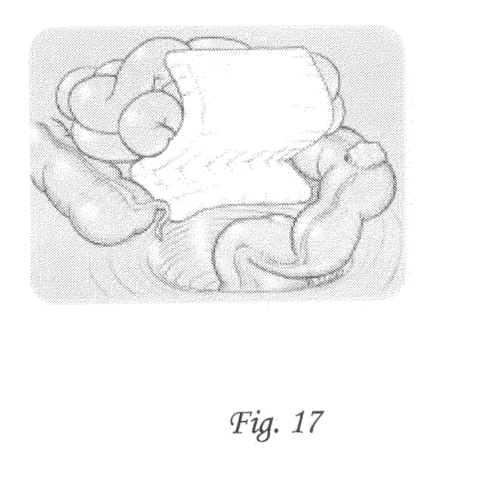

FIG. 17 illustrates the use of the present invention as a retractor during sigmoidectomy. In this operation, the device—originally shaped as rectangle—is sculpted by the surgeon into an S or Z shape, after which the intestines are then placed on the “shelf” created by the upper part of the S shape. Pressure created by the intestine on the upper part of the device is balanced by the lower part of the device, which is pressured by the heavier upper part, thereby pushing it toward the intestine. In doing so, the device is positioned as a barrier between the intestine and the surgical field with no need for extra support. The malleable characteristics of the plastically deformable element allow the surgeon to continually shape and reshape again the device as well as parts of the device, to accommodate anatomical, retraction, and stability needs as the surgical procedure demands. The use of a pad of the invention bent into an S- or Z-shape is to retract the intestines during abdominal surgery is also shown in FIG. 13.

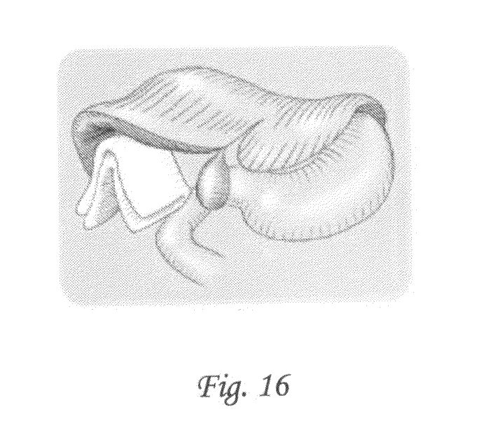

FIG. 16 depicts the use of a folded pad device of the present invention in order to create a surgical field allowing access to the gall bladder and bile duct.

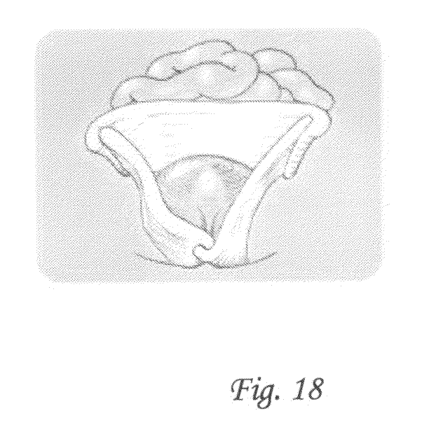

In the embodiment presented in FIG. 18, wherein three separate devices of the present invention are used together in urological surgery, such that the assembly of said three separate units is able to clearly define a self retained surgical window

Another method of use of the plastic form of the invention is when the pad serves as a protective barrier (either in addition to, or instead of, the device's role as a surgical retractor). During the course of surgical procedures, the accidental injury of vessels and other vital organs by the surgeon is a universal risk. The common approach to addressing this problem is by the surgeon locating and marking the position of the vessel within the surgical area. In many instances, this approach is not sufficient. The marking is sometimes unnoticeable, due to fluids or bad visualization in the surgical field.

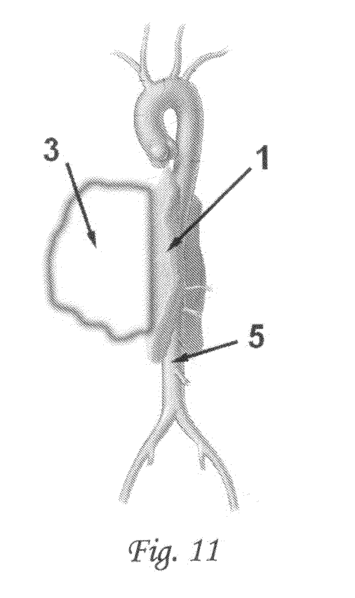

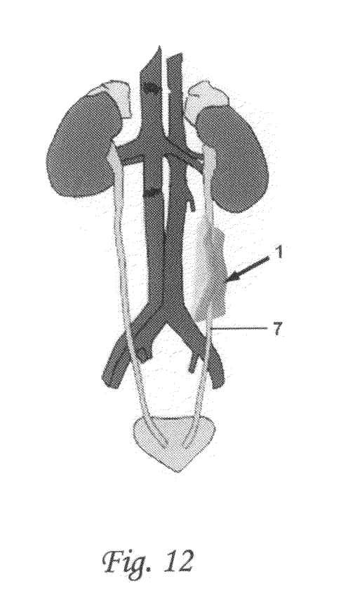

The device of the present invention provides a new solution: the pad of the invention is shaped and inserted around the organ or blood vessel in question, in order to protect said organ or vessel from unintentional injury. In use, once the surgeon has reached the surgical field and located the vessels in proximity to the surgery site, he places the pad on top of the vessels and folds the pad's distal end, wrapping the vessel on all sides. Both the inner rigid deformable plastic element and the layers of the absorptive element secure and protect the vessel from accidental injury. FIG. 11 schematically illustrates the device 1 covering and protecting major blood vessels 5 including the aorta, as well as creating a clear surgical field 3. FIG. 12 schematically illustrates the use of the device 1 in order to cover and protect the ureter 7.

IN THE ELASTIC EMBODIMENT OF THE INVENTION, the deformable core component of the device is comprised of elastically deformable elements, for example in the form of strips. In one preferred embodiment, the strips are placed at a mutual spacing of 3-10 mm, lengthwise, or crosswise, or both lengthwise and crosswise (as shown in FIGS. 1 through 4.) The longitudinal and horizontal lines may or may not be attached to one another to form a net. The strips are then covered on all sides with absorptive material, for example as sponge or gauze pad. In an alternative embodiment, the elastically deformable matrix is surrounded on all sides by a non-absorbent pad or sheet.

The absorptive element, when used, provides the device with the ability to absorb fluids from the surrounding operating area. In addition, the pad serves as a cushion for the incision and surrounding surgical area, protecting it against injury from the surgical tools and massive retractors in use to open the abdominal wall. The inner deformable elastic core component gives the device its ability to be shaped and reshaped in multiple, three dimensional, directions. The elastic configuration forces the element to expand to its maximal, resting state, position, and to remain steady between tissues (for example: the abdominal wall), allowing retraction and\or tissue protection with accordance to the specific variable anatomical configuration and the surgeon's operative needs.

These characteristics provide the device with its ability to act as a “stand alone” (hands-free) pad retractor with no need for securing elements attached to an external frame.

The above-described elastically-deformable embodiment may also be produced in several different forms. Thus, the lines formed by the matrix may be straight (FIG. 5A), curved, wavelike (FIG. 5C and FIG. 5B), curled (FIG. 5E), or any combination of the above (FIG. 5D),), and in any geometric shape.

The elastic deformable element may be produced as a plate or as a plate with lines of holes, both longitudinal and horizontal. The holes may be in various sizes, or may all be one size (FIG. 8). The holes can be made, for example, using laser cutting technology.

The elastically-deformable matrix may be formed in any geometric shape, and size. Some examples are provided in FIG. 6, demonstrating a cross-section view of some of the more commonly used geometric shapes.

EXAMPLES OF USE OF THE ELASTIC EMBODIMENT: This embodiment is intended for retraction of tissues and organs, allowing for maximization of the surgical field, as well as securing the surgical area from injury by way of cushioning the relevant organs and vessels. Another aim of certain preferred embodiments is to absorb biological fluids such as blood. All of this can be accomplished without the need for an outside securing force. The elastic characteristic of the invention allow it to naturally remain in an open position.

FIG. 10 is an illustration of one example of the invention in its open position. The invention 1 is positioned perpendicular to the surgical area 3. The device may be inserted between the two vertical abdominal walls, creating a “wall” perpendicular to the abdominal wall, parallel to the intestine. The device then supports itself in this position against the abdominal walls, by nature of the inner elastically deformable element, in which, when in close proximity to the abdominal wall, both distal sides of the pad lock the device into position. The device thus serves as a barrier preventing the intestine from collapsing into the surgical field.

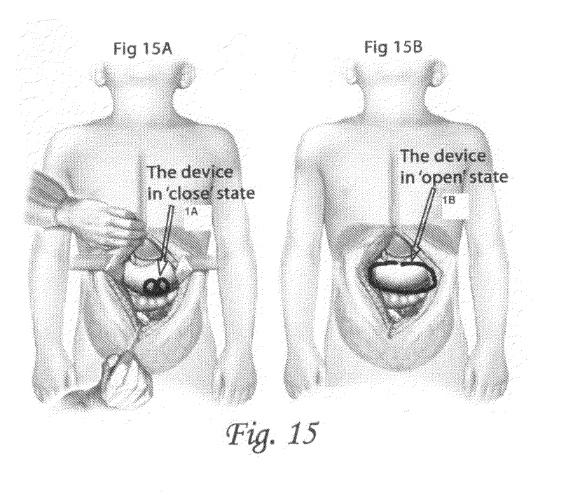

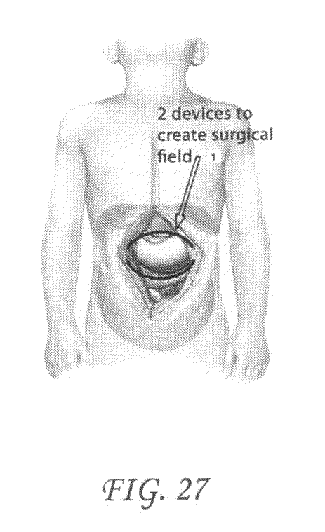

Another exemplary use of the invention is in Cesarean sections (“C-sections”). C-sections require opening and retraction of the lower abdominal wall, as well as securing the proximity of the incision by means of retraction and cushioning. After making the required incision in the abdominal wall, the surgeon would place a few regular abdominal retractors, in order to create an appropriately sized ‘surgical field.’ The pad of this invention, in its semi-open position, could then be placed parallel to the resultant hole in the abdominal wall. The pad would then be opened at the edge of the abdominal wall, creating a well-defined surgical field, and serving as a barrier for, and cushioning, the incision site. Once the pad of this invention is in place, the surgeon may remove the regular, more massive retractors, and in doing so, would allow for more working space for herself, and more safety for the fetus. FIG. 15A illustrates the invention in its closed position 1A, before placement into the abdominal wall. FIG. 15B illustrates the invention in its open state 1B positioned at the upper side of the abdominal wall, maintaining the abdominal wall open. Similarly, FIG. 27 illustrates the use of two elastic embodiment devices 1 of the present invention to create an isolated surgical field during a Cesarian section procedure.

CONSTRUCTION OF THE PLASTICALLY OR ELASTICALLY DEFORMABLE ELEMENT: The device may be constructed from a single type of material, or from a plurality of different types of materials, exhibiting the physicochemical property and behavior of plasticity OR elasticity, whereby the device, in general, and, at least one plastic component OR elastic component, in particular, are deformable. For example, such material can either be selected from a pure metal, a metal alloy, plastic, polymer, wax or any combination thereof. Exemplary pure metals are tungsten, platinum, and titanium. Exemplary metal alloys are nitinol and stainless steel. One particularly preferred type of stainless steel is stainless steel AISI 304. The diameter of each of the deformable element (strips) is generally in the range of 0.5 mm to 5 mm, The surface area of the deformable element is generally in the range of 30×30 mm to 500×500 mm. The length and width of each of the deformable elements is in generally of the same order as the length and/or width of the outer absorptive element (see below).

In one of the deformable element embodiments, the element is made of a single plate (not strips). Generally the surface size of the plate would be in the range of 30×30 mm to 500×500. The plate thickness is generally 0.2 to 5 mm. Suitable materials for constructing such a plate include (but are not limited to) stainless steel (e.g. types 316 and 316L, 304, PH17/4) or a polymer such as polycarbonate.

For example, as an integral, single, continuous plastic component, the device may be designed, configured, and constructed by starting with a single, unitary, preferably metal plate, followed by removing, for example, by laser cutting, selected material from the plate, until only the desired geometry, shape, and dimensions remain. Another method for the construction of the device is by adjoining wire or multiple wires into the desired shape. Industrial bending machinery may be used to bend the wire into the desired shape, and the wires may be connected by welding, in cases where multiple wires are used. For example, metal wires may be obtained from Allvac, Inc. in Monroe, N.C.

The physical properties of the core component of the device—particularly elasticity and plasticity—are determined both by the material selected for use in construction of the core and also by pre-treatment of the material. Thus, for example, maximal elasticity of a given material (e.g. Stainless steel AISI304) is obtained by a cold work hardening process. On the other hand maximal plasticity (for the same material) may be obtained by means of subjecting the core component to a high temperature annealing process.

In one preferred embodiment, the core component is annealed at 1050 deg. Celsius for a period of 0.5 hours, in order to impart the required plasticity on the core component material. Thus, in one preferred embodiment of the device of the invention, the core component of said device is a stainless steel matrix that possesses plasticity equivalent to the plasticity that could be obtained by annealing the same matrix at 1050 deg. Celsius for a period of 0.5 hours.

In the case of the elastic embodiments of the present invention the core component is a matrix that may either be cold-worked or subjected to heat treatment. In a preferred embodiment of the elastic version of the invention, the matrix may be made of stainless steel PH 17-4. In this embodiment of the invention, the hardening mechanism is a specific heat treatment. In a particularly preferred embodiment, the matrix is subjected to an ‘H900’ treatment regime, i.e. 900 degrees Fahrenheit (450 degrees Celsius) for a period of one hour. Such treatment leads to the development of maximal elasticity in the matrix. In other embodiments, the matrix may be subjected to H925 or H1025 or H1075 treatment regimes (i.e. at a Fahrenheit temperature equivalent to the stated number, for a period of one hour), in order to obtain sub-maximal levels of elasticity. As mentioned hereinabove, the matrices of the present invention may also be rendered elastic by means of a cold hardening process. In such cases maximum elasticity may be obtained using cold hardened stainless steel 304 or 316, or another biocompatible stainless steel. In other embodiments, less than maximal elasticity may be obtained by using other materials and cold treatment regimes, e.g. 50% cold-worked stainless steel 304. By means of selecting the appropriate heat or cold-treatment regimes, matrices having specific elastic properties may be prepared.

As mentioned hereinabove, the core element of the device of the present invention may be constructed from several different materials and may be treated in a variety of ways in order to attain varying degrees of plasticity or elasticity. However, it has been found by the present inventor that certain degrees of plasticity or elasticity are more desirable than others from the point of view of the functionality of the device as a hands-free surgical retractor. In order to be able to quantify the degree of plasticity or elasticity, two technically-simple tests were developed:

### 1. Plasticity Test:

This test measures the ability of the plastic embodiments of the device of the present invention to be deformed in a plastic manner (i.e. to retain their deformed conformation after the removal of the manually-applied forces that caused said deformation). The basis of this test is the fact that devices having higher degrees of plasticity will deviate less from their deformed conformation after removal of the deforming force (i.e. manually-applied pressure), while less plastic devices will deviate more from said conformation. In this test, the degree of plasticity is thus expressed as an angular deviation from the deformed conformation, defined by the inventor as the rebound angle (RA). The test is performed as follows:
a) The plastically-deformable core component (or completed pad device comprising both core component and pad) is placed vertically, with its lower half in apposition to the vertical portion of a right-angled former (e.g. the vertical side of a work-bench).
b) The upper half of the core component or device to be tested is then manually deformed such that it is brought into contact with the upper, horizontal portion of said right-angled former (e.g. the top surface of a work-bench). Thus, when the vertical and horizontal portions of the deformed core component or device are firmly held against the corresponding surfaces of the right-angled former (e.g. work-bench), said portions are disposed with an angle of 90 degrees therebetween.
c) The horizontal portion of the core component or device is then released from being held against the horizontal surface of the former, while the vertical portion is still firmly held against the vertical surface of the former.
d) In most cases, following release of the horizontal portion of the core component or device, said portion will rebound slightly upwards and away from the horizontal surface of the former. The angle between the released previously-horizontal portion of the device or core component and the horizontal surface of the former is either measured directly, or calculated by trigonometric means. This angle is defined as the rebound angle (RA).

In one preferred embodiment of the invention, the core component has an RA value between about 6 and about 32 degrees. In a more preferred embodiment, the RA value falls within the range of about 9 to about 20 degrees. In a particularly preferred embodiment, the RA value of the core component is about 12.5 degrees.

### 2. Elasticity Test:

This test measures the ability of the elastic embodiments of the device of the present invention to be deformed in an elastic manner (i.e. to return to their initial conformation after the removal of the manually-applied forces that caused their deformation). The basis of this test is the fact that after having been deformed and then released, devices having higher degrees of elasticity will adopt a conformation more similar to their initial conformation than less elastic devices, the latter devices demonstrating a more marked deviation from said initial (i.e. resting) conformation. In this test, the degree of elasticity is thus expressed as an angular deviation from the original resting conformation following deformation and subsequent release, said deviation being defined by the inventor as the elastic deviation angle (EDA). In its most general form, the test is performed as follows:
a) The core component to be tested is manually folded or bent such that said core component is brought from its initial resting state to a second, elastically deformed state;
b) The manual force used to fold or bend said core component is then removed such that said core component elastically returns to a state close to its initial, resting state;
c) The angle between a selected region of the core component when at the end of step (b) and the position that the same portion adopted when in its initial, resting state is measured or calculated (e.g. by trigonometric methods), said angle being defined as the EDA of said core component.

It is to be recognized that there are several different possibilities regarding the conformations that the various core components of the present invention may adopt in their initial, pre-deformation state. Thus, some elastic devices may be designed and constructed such that in their initial state they have an arcuate profile, while others may be folded in various ways. In one preferred embodiment, the elastic core element is in the form of a flat rectangle or square in its initial state. In the case of this particular embodiment, the EDA determination described immediately hereinabove may be carried out as follows:
a) The flat elastic core component (or complete pad device) to be tested is placed on a flat surface, such as a work-bench top.
b) The core component or device is then folded over on itself, such that approximately half of the device is firmly held on to the flat surface, while the other, second half is brought through an angle of 180 degrees such that its upper surface comes to rest on the upper surface of the half of the core component (or device) that is still being firmly held on the flat surface.
c) The second (folded) half of the core component or device is then released such that it springs back towards its initial, flat conformation.
d) The angle formed between the lower face of the now-released second half of the core component (or device) and the flat surface (e.g. the work-bench top) is then either measured directly or calculated trigonometrically. This angle is defined as the elastic deviation angle (EDA).

In one preferred embodiment of the present invention, the core component has an EDA value within the range of about 0 to about 50 degrees. In another, more preferred embodiment, the core component has an EDA value in the range of about 0 to about 20 degrees. In a still more preferred embodiment, the core component has an EDA value of about 7 degrees.

Regardless of whether the core component element is plastic or elastic, or whether it is constructed from an array of wires or from a perforated sheet, the presence of the pores, spaces or holes in the core is very important from the point of view of permitting transfer (seepage) of fluids such as blood from one face of the device to the other. It will be readily appreciated that if seepage is unable to occur (e.g. if the core component is non-porous) or if it occurs at too low a rate (e.g. if there is an insufficient number of pores and/or the mean pore size is too small), the liquid absorbing capacity of the device (taken as a whole) will be dependent to a large extent on the absorptive capacity of the gauze pad on one side only of the device (i.e. the side in contact with the blood or other fluid). However, in the event that the core component is sufficiently porous, blood and other fluids may be absorbed by one face of the gauze pad and by a process of seepage through said pores enter the other gauze pad face and be absorbed thereby.

The ability of a core matrix to permit seepage may thus be seen to be a function of the pore density of said matrix. In addition to its relation to fluid seepage, the pore density of the matrix of the present invention also influences the overall strength of the device and (in the case of the plastic embodiment) the plasticity of the device.

For the purpose of the present discussion, core component pore density may be defined as the total area occupied by pores or holes divided by the total area of the core component. For practical purposes this may be calculated as:

{(P−D)̂2*Nx*Ny}/{(Nx*P+D)*(Ny*P+D)}

Wherein:
D: wire diameter
Nx: number of holes in ‘X’ direction
Ny: number of holes in ‘Y’ direction
P: pore pitch

By way of example, the core component (‘matrix’) pore density of an exemplary matrix of the present invention was calculated to be 84.7%. However, matrices having pore densities both lower and higher than this figure may also be used to construct the pad retractor of the present invention. The preferred range of matrix pore density is about 10% to about 95%. A more preferred range is about 50% to about 90%. In a particularly preferred embodiment, the matrix pore density is about 85%.

An exemplary pad device of the present invention having the abovementioned preferred matrix pore density of about 85% was tested in order to determine the ability of the device to promote seepage of liquid applied to one face of the pad to the other face thereof. Briefly, the pad was supported in a horizontal position by means of a retort clamp. A colored liquid was then caused to drip from above onto the upper face of the device at a rate of 3.3 drops per second, and the time taken for the colored liquid to be visible on the lower face of the pad is recorded. Following a total of five such measurements (with a fresh device each time), the mean time taken for the liquid to appear on the lower face was calculated as 33.6 seconds.

OUTER ABSORPTIVE OR NON-ABSORPTIVE SHIELDING ELEMENT: In all embodiments of the device, the plastically OR elastically deformable core component is coated by a protective pad or sheet. In one preferred embodiment, the pad or sheet is constructed of an absorbent material such as gauze, preferably constructed of three to six ply, absorbent, non-shedding cotton gauze. Other suitable materials other than cotton gauze such as non woven cotton may be used if desired. In another preferred embodiment, the protective pad is constructed from a non-absorbent material including (but not limited to) polymeric materials such as polyurethane, polyester, polyamide and silicon coated textiles or sheets. In one preferred embodiment, the absorptive pad is constructed from 100% cotton gauze.

As explained hereinabove, the softness of the outer pad component is an important factor in determining the non-traumatic nature of the device. The softness of one preferred embodiment of the device of the present invention was tested in the following manner:
1. A pad retractor comprising a stainless steel matrix with soft silicon tubing covered ends, encased within two cotton gauze pouches, each pouch consisting of 6 layers of 100% cotton gauze, was constructed.
2. The pad retractor was placed on a hard work surface and a 30 mm diameter metal ball having a mass of 100 g was then placed on said retractor.

3. Using a test device that provides accurate measurement of vertical distances, depth of the depression formed by the ball in the gauze pad was measured.

Seven such tests were performed, and the mean depression caused by the ball was found to be 12±1 mm. The indirect measurement of softness provides an exemplary indication of the degree of pad softness that is required for the device of the present invention. The abovementioned result should not, however, be regarded as limiting in any way.

In addition to its function as a soft barrier, the outer gauze pad component, as explained hereinabove, is also responsible for the fluid absorption capability of the presently-disclosed device. This capability is essentially defined by two different parameters: the total absorbing capability of the device and the rate of absorbance by the device. In one preferred embodiment of the invention, the total absorbing capability of the device was found to be 700 ml, while the rate of absorbance was measured as approximately 230 ml per second. These results should, however, be understood only as exemplary in nature, and do not limit the invention in any way, since other embodiments of the invention possessing higher or lower absorbance parameters are also to be considered as forming part of the present invention as disclosed herein.

SIZE OF THE DEVICE: The length of the inner plastically deformable element or elastic element would be similar to the length and/or width dimensions of the outer absorbent gauze pad, or smaller in size than the outer absorbent gauze pad. The device may be manufactured to every size of pad and surgical drape commonly found in a surgery room. Most commonly the device will have surface size of 200×150 mm to 300×200 mm, though the device may be manufactured in any size or thickness as commonly in use for pads, sponges and surgical drapes.

### Exemplary Manufacturing Approaches:

### First Exemplary Manufacturing Approach:

1. Matrix

The matrix (depicted in FIG. 20) is made of heat treated stainless steel that provides the device with its plasticity characteristics. The matrix shown in FIG. 20 was constructed by welding a series of horizontal and vertical wires. In an alternative embodiment, the matrix can be manufactured as a woven mesh.

2. Silicon Tubing

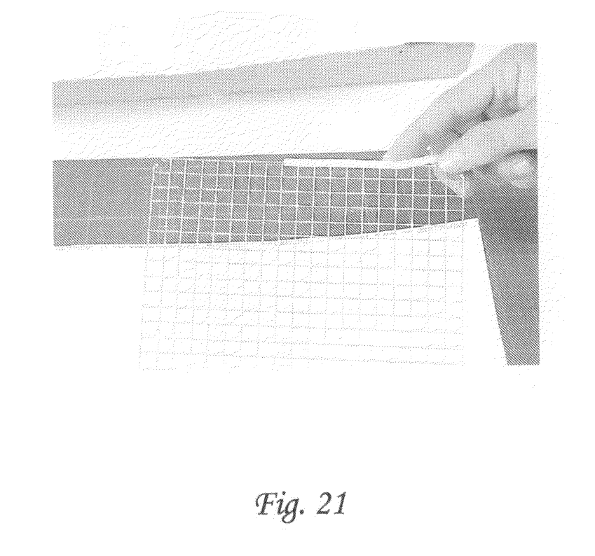

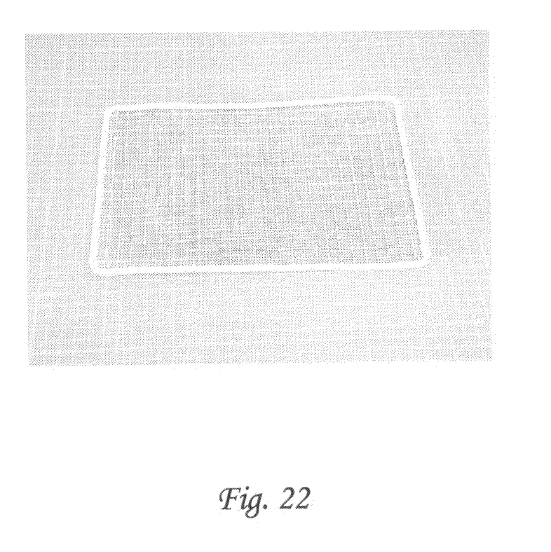

A length of silicon tubing is glued around the border of the matrix, as shown in FIGS. 21 and 22. The purpose of the Silicon tubing is to cover any sharp edges, giving the matrix ends their soft touch. Although any suitable tubing may be used for this purpose, particularly preferred tubing is 2.5 mm thick edge silicon tubing having 50 to 60 Shore (scale ‘A’) hardness.

3. Pouches

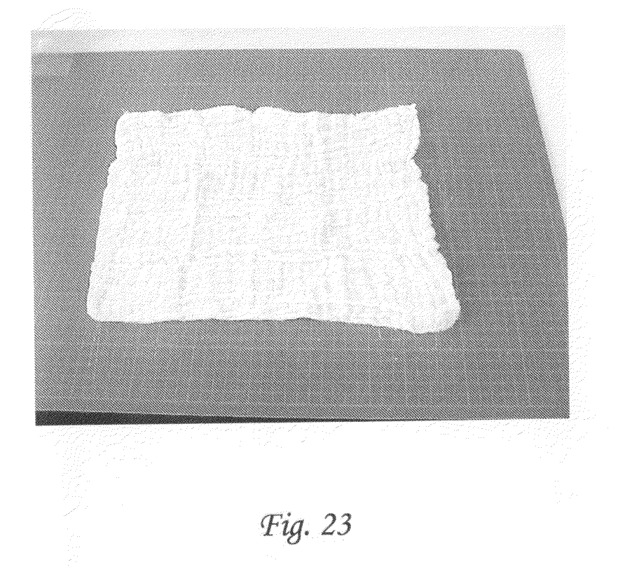

The matrix is covered by internal and external pouches. The pouches consist of layers of gauze pad (see FIG. 23). The gauze pad gives the device its padding and absorptive behavior. Furthermore, together with the peripheral silicon tubing described hereinabove, the soft pad reduces or prevents the potentially damaging application of undesirably-high mechanical pressures to small areas of the retracted tissues and organs, thereby reducing the chance of iatrogenic trauma.

4. The Internal Pouch

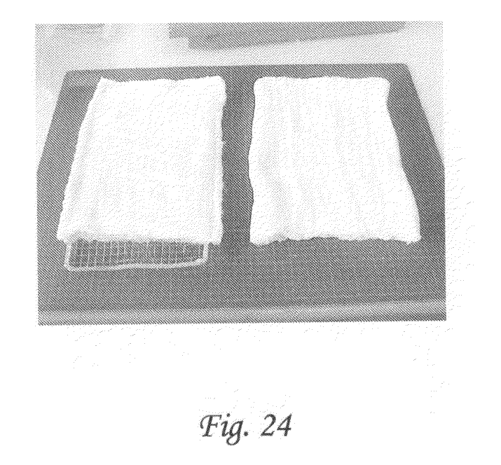

The internal pouch consists of 6 layers of gauze pad (see FIG. 24) sewn by cotton thread

5. The External Pouch

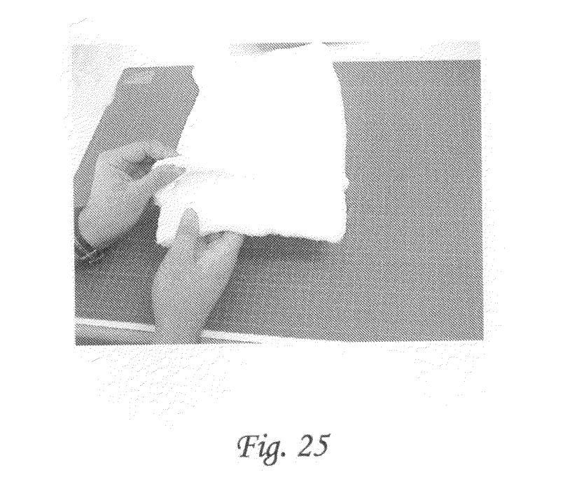

The external pouch is the outside padding of the device. Similar to the inside pouch, it consists of 6 gauze pad layers (see FIG. 25) sewn to one another by means of cotton thread.

6. Final Sewing

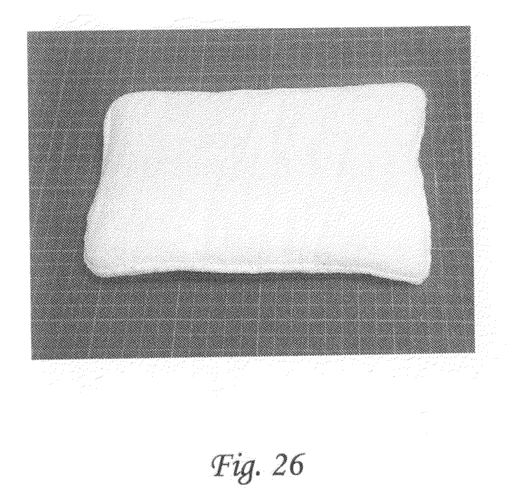

The final step in the manufacture of the device is additional sewing around the entire border of the device to strengthen the layer assembly (see FIG. 26). Following this, the device is packaged.

Second Exemplary Manufacturing Approach:
Step 1—a strip of wire of the desired diameter is cut to form the desired length.
Step 2—similar strips are cut as needed to form the desired shape, width and length of the pad.
Step 3—The distal and proximal ends of the wires are smoothed in order to avoid rupture of the absorptive element. As an alternative to mechanical smoothing, the wire matrix may be coated with polymeric materials by means of high pressure injection. A similar result may also be achieved by adhering silicon tubing around the margins of the wire matrix using, for example, silicon glue.
Step 4—the lines of strips are placed on top of a ply of the absorptive material in the preferred structure as shown in FIGS. 1 to 5.
Step 5—the lines of wire are secured to the absorptive material by suturing or gluing.
Step 6—one or more further layers of the absorptive material are placed over the wire matrix.
Step 7—The lower and upper parts of the plies (layers) are attached to one another by means of suturing or gluing.

### Third Exemplary Manufacturing Approach:

Step 1—The plate is cut and shaped to the desired dimension and geometric shape. Cutting is performed by laser or by means of chemical etching techniques or by means of mechanical techniques such as punching.
Step 2—Holes of the desired shape and/or diameter are cut, for example by a laser technique, on the edges of the plate on all sides, with a space (e.g. 10 mm) between each hole.
Step 3—The plate is placed on top of a ply of the absorptive element and secured to same by means of suturing, using the created holes.
Step 4—A second ply of the absorptive element is placed on the upper side of the plate and secured as described above.
Step 5—The two sections of absorptive element—the lower and upper—are then attached by means of suturing or gluing.

Following manufacture, the device may be sterilized by means commonly in use in the industry, including (but not limited to) ethylene oxide, gamma, and electron-beam radiation.

It is to be noted that the deformable pad device of the present invention is also capable of being used for non-surgical purposes. One such non-surgical application is the use of the deformable pad as a packaging material, in order to provide both mechanical protection for packaged articles, as well as immobilization within a carton or other container as a result of the forces exerted by the plastically-deformable or elastically-deformable core component.

While specific embodiments of the invention have been described for the purpose of illustration, it will be understood that the invention may be carried out in practice by skilled persons with many modifications, variations and adaptations, without departing from its spirit or exceeding the scope of the invention as defined and claimed herein.

1. A pad device comprising a plastically deformable or elastically deformable core component embedded within a pad, sheet or drape.

2. The pad device according to claim 1, wherein the core component is plastically deformable.

3. The pad device according to claim 1, wherein the core component is elastically deformable.

4. The pad device according to claim 1, wherein the deformable core component is present across the entire width and length of the pad.

5. The pad device according to claim 1, wherein the deformable core component occupies less than 100% of the area of the pad.

6. The pad device according to claim 1, wherein the pad is an absorbent gauze pad.

7. The pad device according to claim 1, wherein the deformable core component is a matrix comprising a series of parallel strips.

8. The pad device according to claim 1, wherein the deformable core component is a matrix comprising two series of parallel strips, wherein one series is disposed at an angle of up to 90 degrees to the second series.

9. The pad device according to claim 1, wherein said device is capable of being manually deformed in any desired direction.

10. The pad device according to claim 1, wherein the weight of the pad is at least 35% of the total weight of the device.

11. The pad device according to claim 1, wherein the weight of the pad is at least 70% of the total weight of the device.

12. The pad device according to claim 2, wherein the core component of said device is a stainless steel matrix that possesses plasticity equivalent to the plasticity obtained by annealing the same matrix at 1050 deg. Celsius for a period of 0.5 hours.

13. The pad device according to claim 2, wherein the core component of said device is characterized by having a rebound angle (RA) in the range of about 6 to about 32 degrees, wherein said RA is calculated by a method comprising the steps of:
a) providing a right-angled former having a vertical surface and a horizontal surface;
b) bending the core component to be tested across said right-angled former, such that said core component acquires a vertical surface and a horizontal surface corresponding to the surfaces of said right-angled former, wherein said core component vertical surface and said core component horizontal surface form an angle of 90 degrees therebetween when said surfaces are firmly held on said right-angled former;
c) releasing the horizontal surface of said core component, while the vertical surface thereof is still held firmly against the corresponding surface of the right-angled former, thereby permitting the free end of said core component horizontal surface to move out of contact with the horizontal surface of said former; and
d) measuring or calculating the angle formed between the released core component horizontal surface and the horizontal surface of the right-angled former, said angle being defined as the RA of said core component.

14. The pad device according to claim 13, wherein the core component of said device is characterized by having an RA in the range of about 9 to about 20 degrees.

15. The pad device according to claim 14, wherein the core component of said device is characterized by having an RA of about 12.5 degrees.

16. The pad device according to claim 3, wherein the core component of said device is characterized by having an elastic deviation angle (EDA) in the range of about 0 to about 50 degrees, wherein said EDA is calculated by a method comprising the steps of:
a) folding or bending the core component to be tested such that said core component is brought from its initial resting state to a second, elastically deformed state;
b) removing the manual force used to fold or bend said core component such that said core component elastically returns to a state close to its initial, resting state;
c) measuring or calculating the angle between a selected region of the core component at the end of step (b) and the position that the same portion adopted when in its initial, resting state, said angle being defined as the EDA of said core component.

17. The pad device according to claim 16, wherein the core component of said device is characterized by having an EDA in the range of about 0 to about 20 degrees.

18. The pad device according to claim 17, wherein the elasticity of the core component of said device is characterized by having an EDA of about 7 degrees.

19. The pad device according to claim 3, wherein said device has an essentially flat conformation when in its initial, resting state, and wherein the core component of said device is characterized by having an elastic deviation angle (EDA) in the range of about 0 to about 50 degrees, wherein said EDA is calculated by a method comprising the steps of:
a) placing the core component to be tested on a flat surface;
b) folding said core component approximately in half such that the core component is brought from an open, flat conformation into a closed conformation with the lower face of a first half of said device remaining in contact with said flat surface, the other, second half of the device being folded through an angle of approximately 180 degrees such that its upper face is brought into contact with the upper face of said first half;
c) releasing said second half of the pad device such that the device tends to return to its previous open flat conformation; and
d) measuring or calculating the angle between the lower face of said second half and said flat surface, said angle being defined as the EDA of said core component.

20. The pad device according to claim 19, wherein the core component of said device is characterized by having an EDA in the range of about 0 to about 20 degrees.

21. The pad device according to claim 20, wherein the elasticity of the core component of said device is characterized by having an EDA of about 7 degrees.

22. The pad device according to claim 1, wherein the core component has a pore density of about 10% to about 95%.

23. The pad device according to claim 22, wherein the core component has a pore density of about 50% to about 90%.

24. The pad device according to claim 22, wherein the core component has a pore density of about 85%.

25. The pad device according to claim 1, wherein said device is biocompatible and sterilizable.

26. A method for hands-free retraction of organs and tissues during surgery comprising the steps of:
a) providing a pad device according to claim 25;
b) bending the pad device to the desired shape; and
c) inserting the pad device into the desired location within the surgical field, thereby causing the retraction of the tissues and organs present at said location;
wherein steps (b) and (c) may be performed in any order.

27. A method for protecting organs and tissues during surgery comprising the steps of:
a) providing a pad device according to claim 25;
b) bending the pad device to the desired shape; and
c) positioning the pad device around the organ or tissue to be protected;
wherein steps (b) and (c) may be performed in any order.

28. The method according to claim 27, wherein the organ or tissue to be protected is a blood vessel.

29. A method for hands-free retraction of soft tissues during dental procedures comprising the steps of:
a) providing a pad device according to claim 25;
b) bending the pad device to the desired shape; and
c) inserting the pad device into the desired location within the oral cavity, thereby causing the retraction of the tissues present at said location;
wherein steps (b) and (c) may be performed in any order.
